(19) United States
(12) Patent Application Publication
(10) Pub. No.: US 2025/0246272 A1
(43) Pub. Date: Jul. 31, 2025

(54) GRADIENT BOOSTING DECISION TREE PREDICTION METHOD FOR SANDSTONE DRILLABILITY BASED ON CRYSTAL STRUCTURE AND MINERALOGICAL CHARACTERISTICS

(71) Applicant: SOUTHWEST PETROLEUM UNIVERSITY, Chengdu (CN)

(72) Inventors: Xiangchao SHI, Chengdu (CN); Shuai CHEN, Chengdu (CN); Yuming WANG, Chengdu (CN); Wenqiang XIAO, Chengdu (CN); Yan CHEN, Chengdu (CN); Zhaowei WANG, Chengdu (CN); Haode DUAN, Chengdu (CN)

(21) Appl. No.: 18/829,422

(22) Filed: Sep. 10, 2024

(30) Foreign Application Priority Data
Jan. 31, 2024 (CN) 202410133118.5

(51) Int. Cl. G16C 60/00; G01N 33/00; G16C 20/70

(52) U.S. Cl. CPC G16C 60/00; G01N 33/39; G16C 20/70

(57) ABSTRACT

Disclosed is a gradient boosting decision tree (GBDT) prediction method for sandstone drillability based on crystal structure and mineralogical characteristics, including: acquiring cuttings samples of an area to be tested, dividing crystal boundaries based on the cuttings sample, and acquiring a plurality of crystal samples; numbering the plurality of the crystal samples, and extracting geometric parameters and mineral components of the plurality of the crystal samples; performing a correlation analysis on the geometric parameters, the mineral components and drillability data to obtain geometric parameters, the mineral components and the drillability; dividing the geometric parameters, the mineral components and the drillability into a training set and a testing set; training a GBDT model through the training set to obtain a trained GBDT model; and detecting accuracy of the trained GBDT model through the testing set to obtain prediction accuracy of trained GBDT model.

### CROSS-REFERENCE TO RELATED APPLICATIONS

This application claims priority to Chinese Patent Application No. 202410133118.5, filed on Jan. 31, 2024, the contents of which are hereby incorporated by reference.

### TECHNICAL FIELD

The disclosure belongs to the technical field of sandstone fragmentation in petroleum drilling engineer, and particularly relates to a gradient boosting decision tree (GBDT) prediction method for sandstone drillability based on crystal structure and mineralogical characteristics.

### BACKGROUND

In oil and gas drilling engineering, sandstone drillability has always been the most concerned parameter for bit design engineers. At present, the most commonly used method to measure the sandstone drillability is the laboratory micro-drilling test method, but there are problems such as difficult coring and high drilling cost, and it is extremely difficult to obtain the drillability index of the whole well section. Sandstone drillability affects the life of drill bit, drilling cycle and drilling cost. Accurate prediction of sandstone drillability is of great significance in improving drilling efficiency, saving drilling cost and reducing drilling cycle. In the prior art, the sandstone drillability is evaluated by using the content of cuttings elements, but the geometric structure of mineral particles in sandstone is not considered, and the prediction model of drillability is not established by using the GBDT method. In the prior art, the sandstone drillability is evaluated by texture coefficient (TC) index based on the drillability characterization method of sandstone texture, yet the influence of mineral differences on drillability is not considered, and the prediction model is not established by using the GBDT method. Therefore, it is urgent to propose a GBDT prediction method for sandstone drillability based on crystal structure and mineralogical characteristics.

### SUMMARY

In order to solve the technical problems, the disclosure provide a GBDT prediction method for sandstone drillability based on crystal structure and mineralogical characteristics, which realize accurate prediction of sandstone drillability through underground cuttings and finally obtains the drillability index of the whole well section.

In order to achieve the above objectives, the disclosure provides a GBDT prediction method for sandstone drillability based on crystal structure and mineralogical characteristics, including the following steps:
acquiring cuttings samples of an area to be tested, dividing crystal boundaries based on the cuttings sample, and acquiring a plurality of crystal samples;
numbering the plurality of the crystal samples, and extracting geometric parameters and mineral components of the plurality of crystal samples;
performing a correlation analysis on the geometric parameters, the mineral components and drillability data to obtain the geometric parameters, the mineral components and the drillability;
dividing the geometric parameters, the mineral components and the drillability into a training set and a testing set;
training a GBDT model through the training set to obtain a trained GBDT model; and
detecting an accuracy of the trained GBDT model through the testing set to obtain a prediction accuracy of the trained GBDT model.

Optionally, a method for dividing the crystal boundaries based on the cuttings samples includes:
making the cuttings samples into slices, and observing the slices through a microscope; and
identifying boundaries between crystals, determining ownership of each crystal, marking the a boundary of each crystal, and obtaining a plurality of crystal samples.

Optionally, geometric parameters of the crystal samples include shape factors, angle factors, areas, diameters and perimeters of the crystals.

Optionally, a method for performing the correlation analysis on the geometric parameters, the mineral components and the drillability data includes:

$$R = \frac{{\sum }_{i}^{n} ( {x}_{i} - \overline{x} ) ( {y}_{i} - \overline{y} )}{\sqrt{{\sum }_{i}^{n} {( {x}_{i} - \overline{x} )}^{2} {\sum }_{i}^{n} {( {y}_{i} - \overline{y} )}^{2}}}$$

where R is Pearson correlation between parameter x and sandstone drillability index y; xᵢ is an i-th sample value of input parameter x; n is a total number of samples; x̅ is an average value of parameters x; yᵢ is a drillability index of the i-th sample; and y̅ is an average drillability index of all the samples.

Optionally, a method for obtaining the geometric parameters, the mineral components and the drillability includes:
sorting the correlation between the geometric parameters and the mineral components and the drillability, and screening geometric parameters and mineral components with high correlation with the drillability as input parameters for establishing a drillability prediction model to obtain the geometric parameters, the mineral components and the drillability.

Optionally, a process of training the GBDT model through the training set to obtain the trained GBDT model includes:
using a decision tree as an initial model for the training set, calculating a residual between a true value of each sample and an initial predicted value, using the residual as a target variable to construct a new decision tree, giving a learning rate, multiplying the learning rate by a predicted value of the new decision tree as a target value increment, adding the initial predicted value and the target value increment to obtain a new predicted value, and using the new decision tree to predict until a predetermined iterations is reached, thus obtaining the trained GBDT model.

Optionally, a method for detecting the accuracy of the trained GBDT model through the testing set includes:
inputting the geometric parameters and mineral components of the testing set into the trained GBDT model to obtain a predicted drillability index, and comparing and analyzing the drillability index of the testing set with the new predicted value to obtain the prediction accuracy of the trained GBDT model.

The disclosure has the following technical effects.

The influence of geometric characteristics and mineral components on drillability is quantified, and the most significant parameters are obtained, which provides theoretical basis for revealing the mechanism of drillability fracture;
the prediction accuracy of sandstone drillability is improved, and with the increase of sample number, the accuracy of prediction model is further improved;
after inputting new data, the results may be quickly calculated and displayed visually;
GBDT algorithm is used to ensure that the model based on the regression between crystal structure and mineralogy characteristics and sandstone drillability will not be over-fitted;
a new method for obtaining the sandstone drillability in the whole well section is provided, which can provide scientific basis for bit design and speed-up tool optimization.

### BRIEF DESCRIPTION OF THE DRAWINGS

The accompanying drawings, which constitute a part of this application, are used to provide a further understanding of this application. The illustrative embodiments of this application and the descriptions are used to explain this application, and do not constitute an improper limitation of this application. In the attached drawings:

FIG. 1 is a schematic flow chart of a GBDT prediction method for sandstone drillability based on crystal structure and mineralogical characteristics in an embodiment of the present disclosure.

FIG. 2 is a prediction model established by using the GBDT algorithm according to an embodiment of the present disclosure.

FIG. 3 is the crystal boundary division result of a sandstone in an embodiment of the present disclosure.

FIG. 4 shows the correlation analysis results of characteristic parameters of an embodiment of the present disclosure.

FIG. 5 is a scatter plot of comparison between predicted sandstone drillability and actual drillability according to an embodiment of the present disclosure.

### DETAILED DESCRIPTION OF THE EMBODIMENTS

It should be noted that the embodiments in this application and the features in the embodiments may be combined with each other without conflict. The present application will be described in detail with reference to the attached drawings and examples.

It should be noted that the steps shown in the flowchart of the accompanying drawings may be executed in a computer system such as a set of computer-executable instructions, and although the logical order is shown in the flowchart, in some cases, the steps shown or described may be executed in a different order from here.

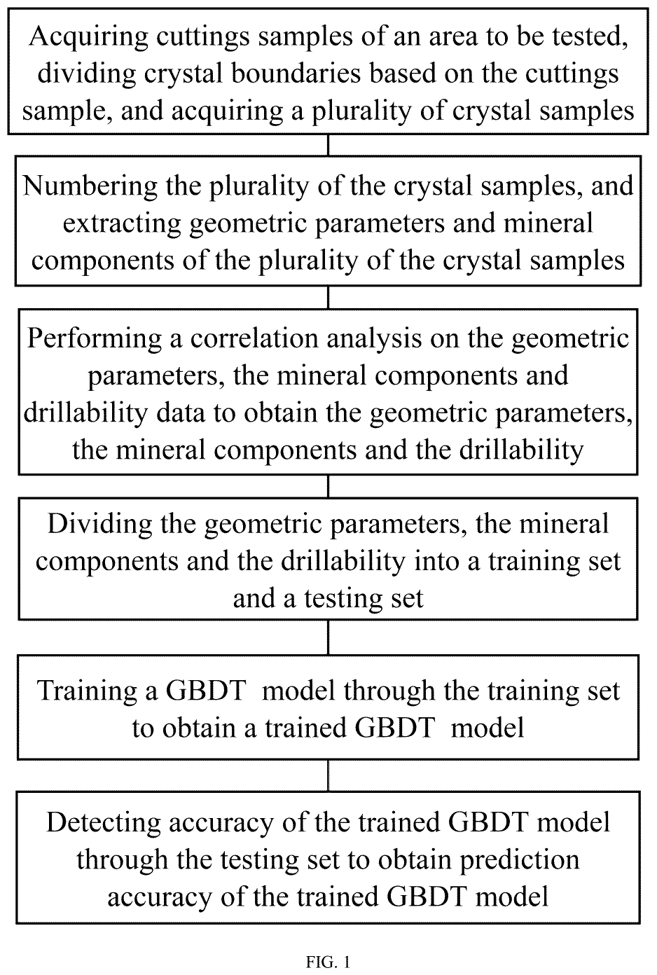

As shown in FIG. 1, a GBDT prediction method for sandstone drillability based on crystal structure and mineralogical characteristics provided in this embodiment includes the following steps:
acquiring cuttings samples of an area to be tested, dividing crystal boundaries based on the cuttings sample, and acquiring a plurality of crystal samples;
numbering the plurality of the crystal samples, and extracting geometric parameters and mineral components of the plurality of the crystal samples;
performing a correlation analysis on the geometric parameters, the mineral components and drillability data to obtain the geometric parameters, the mineral components and the drillability;
dividing the geometric parameters, the mineral components and the drillability into a training set and a testing set;
training a GBDT model through the training set to obtain a trained GBDT model; and
detecting accuracy of the trained GBDT model through the testing set to obtain prediction accuracy of the trained GBDT model.

Dividing crystal boundaries includes identifying the boundaries between crystals, clarifying the ownership of crystals, and marking crystal boundaries with the help of software.

Extracting geometric parameters of particles includes calculating the shape factors, angle factors, areas, diameters, perimeters and so on.

The correlation analysis of characteristic parameters includes the correlation analysis of the extracted geometric parameters and mineral components obtained from XRD experiments and the drillability data obtained from experiments, and the correlation between the extracted geometric parameters and mineral components and drillability is obtained. The calculation expression of Pearson correlation is:

$$R = \frac{{\sum }_{i}^{n} ( {x}_{i} - \overline{x} ) ( {y}_{i} - \overline{y} )}{\sqrt{{\sum }_{i}^{n} {( {x}_{i} - \overline{x} )}^{2} {\sum }_{i}^{n} {( {y}_{i} - \overline{y} )}^{2}}}$$

where R is Pearson correlation between parameter x and sandstone drillability index y; xᵢ is an i-th sample value of input parameter x; n is a total number of samples; x̅ is an average value of parameters x; yᵢ is a drillability index of the i-th sample; and y̅ is an average drillability index of all the samples.

Screening and determining input parameters include sorting the correlation between the geometric parameters and the mineral components and the drillability, screening geometric parameters and mineral components with high correlation with the drillability as input parameters for establishing drillability prediction model, and obtaining geometric parameters and drillability data as a total data set D.

Dividing a training set D1 and a testing set D2 includes randomly dividing the total data set D to obtain the training set D1 and the testing set D2, where the training set D1 accounts for 80% of the total data set D and the testing set D2 accounts for 20% of the total data set D.

The training set D1 is trained by using the GBDT method, and the trained GBDT regression model includes initializing the weak learner, calculating the negative gradient, and updating the strong learner to get the final learner.

Using testing set D2 to test the accuracy of regression model includes using the geometric parameters and mineral components of testing set D2 to bring into the established regression model to get the predicted drillability index, and comparing the drillability index of testing set D2 with the predicted value to obtain the prediction accuracy of regression model, which is used to evaluate the quality of regression model.

Predicting the drillability through the crystal structure and mineralogical characteristics of sandstone includes obtaining the crystal structure and mineralogical characteristics based on underground cuttings, and bringing the crystal structure and mineralogical characteristics into the established GBDT regression model may obtain the drillability index, and finally the sandstone drillability is accurately predicted based on underground cuttings.

In this embodiment, the division of crystal boundaries is the preparation stage of geometric parameter extraction, the extraction of particle geometric parameters is the geometric parameter calculation stage, the correlation analysis and screening of characteristic parameters and determining input parameters are the analysis and screening stages of parameters, and the training set D1 and the testing set D2 are divided into the preparation stage of the GBDT method. Obtaining the trained GBDT regression model and using the testing set D2 to test the accuracy of the regression model belong to the stage of establishing the sandstone drillability prediction model by the GBDT method, and predicting the drillability by the crystal structure and mineralogical characteristics of sandstone is the stage of drillability prediction based on the crystal structure and mineralogical characteristics.

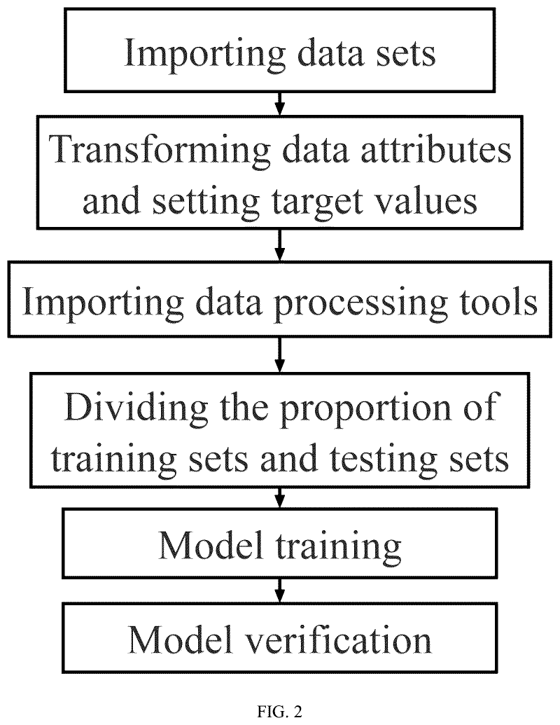

As shown in FIG. 2, the prediction model is established by using the GBDT algorithm, including importing data sets, transforming data attributes and setting target values, importing data processing tools, dividing the proportion of training sets and testing sets, model training and model verification.

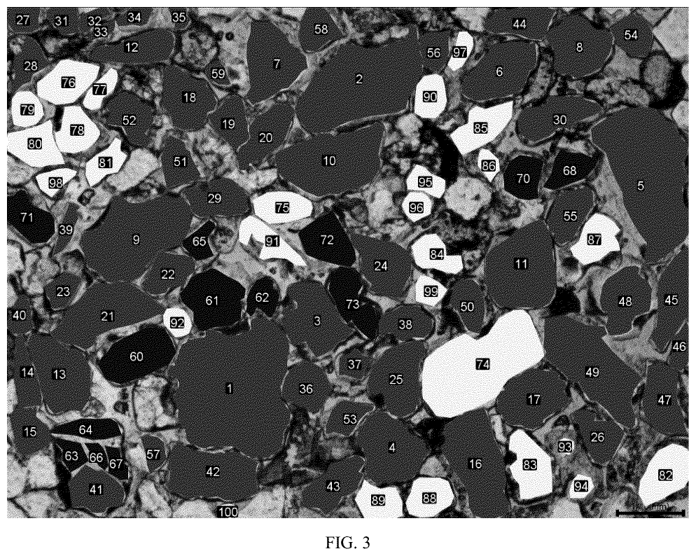

As shown in FIG. 3, the division of crystal boundaries first requires to clarify the adhesion relationship between crystals, then the boundaries of crystals are circled and marked according to the serial number, then all the crystals that may be clearly identified in the microscopic image are circled and marked, and finally the results of crystal boundary division are saved.

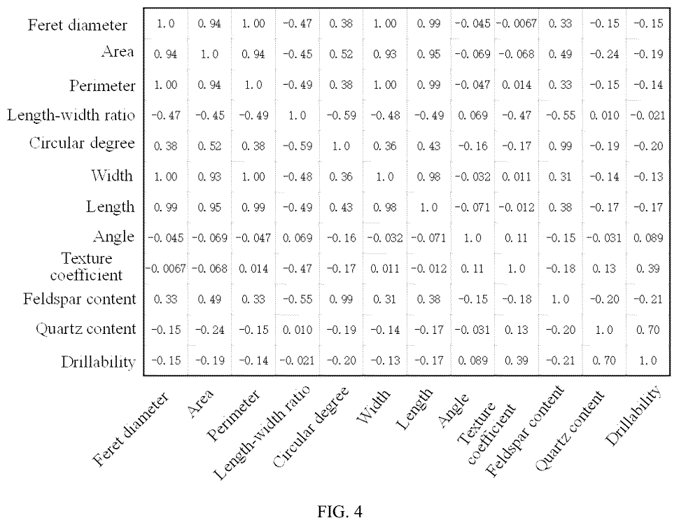

As shown in FIG. 4, the correlation analysis results of feature parameters include: Pearson correlation analysis of Feret, A, p, AR, SF, width, height, angle, TC and Kd, and selecting the parameters with relatively strong correlation with drillability as the input parameters in the data set to improve the efficiency of model training and model prediction based on GBDT prediction method. Feret is the Feret diameter; A is the area; p is the perimeter; AR is the minimum aspect ratio; SF is roundness; width is the width of the smallest circumscribed rectangle; height is the height of the smallest circumscribed rectangle; angle is the included angle between the major axis of the smallest circumscribed ellipse and the horizontal direction; TC is the texture coefficient; and Kd is the drillability index.

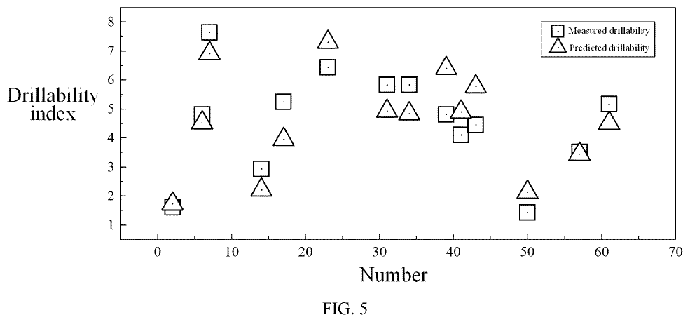

FIG. 5 is a scatter plot of the comparison between the predicted sandstone drillability and the actual drillability of the embodiment. As shown in FIG. 5, the data of the testing set are brought into the established GBDT prediction model of sandstone drillability, and the maximum error between the measured drillability and the predicted drillability is 33.6%, and the average error is 18.1%. The established drillability prediction model has high prediction accuracy.

The above describes only the preferred embodiments of this application, but the protection scope of this application is not limited to this. Any change or replacement that may be easily thought of by a person familiar with this technical field within the technical scope disclosed in this application should be included in the protection scope of this application. Therefore, the protection scope of this application should be based on the protection scope of the claims.

What is claimed is:

1. A gradient boosting decision tree prediction method for sandstone drillability based on crystal structure and mineralogical characteristics, comprising following steps:
acquiring cuttings samples of an area to be tested, dividing crystal boundaries based on the cuttings sample, and acquiring a plurality of crystal samples;
numbering the plurality of the crystal samples, and extracting geometric parameters and mineral components of the plurality of the crystal samples;
performing a correlation analysis on the geometric parameters, the mineral components and drillability data to obtain the geometric parameters, the mineral components and the drillability;
dividing the geometric parameters, the mineral components and the drillability into a training set and a testing set;
training a gradient boosting decision tree model through the training set to obtain a trained gradient boosting decision tree model; and
detecting an accuracy of the trained gradient boosting decision tree model through the testing set to obtain a prediction accuracy of the trained gradient boosting decision tree model.

2. The gradient boosting decision tree prediction method for the sandstone drillability based on the crystal structure and the mineralogical characteristics according to claim 1, wherein a method for dividing the crystal boundaries based on the cuttings samples comprises:
making the cuttings samples into slices, and observing the slices through a microscope; and
identifying boundaries between crystals, determining ownership of each crystal, marking a boundary of each crystal, and obtaining the plurality of the crystal samples.

3. The gradient boosting decision tree prediction method for the sandstone drillability based on the crystal structure and the mineralogical characteristics according to claim 1, wherein the geometric parameters of the crystal samples comprise shape factors, angle factors, areas, diameters and perimeters of the crystals.

4. The gradient boosting decision tree prediction method for the sandstone drillability based on the crystal structure and the mineralogical characteristics according to claim 1, wherein a method for performing the correlation analysis on the geometric parameters, the mineral components and the drillability data comprises:
$$R = \frac{{\sum }_{i}^{n} ( {x}_{i} - \overline{x} ) ( {y}_{i} - \overline{y} )}{\sqrt{{\sum }_{i}^{n} {( {x}_{i} - \overline{x} )}^{2} {\sum }_{i}^{n} {( {y}_{i} - \overline{y} )}^{2}}}$$
wherein R is Pearson correlation between parameter and sandstone drillability index y; xᵢ is an i-th sample value of input parameter x; n is a total number of samples; x̅ is an average value of parameters x; yᵢ is a drillability index of the i-th sample; and ŷ is an average drillability index of all the samples.

5. The gradient boosting decision tree prediction method for the sandstone drillability based on the crystal structure and the mineralogical characteristics according to claim 1, wherein a method for obtaining the geometric parameters, the mineral components and the drillability comprises:
sorting the correlation between the geometric parameters and the mineral components and the drillability, and screening geometric parameters and mineral components with high correlation with the drillability as input parameters for establishing a drillability prediction model to obtain the geometric parameters, the mineral components and the drillability.

6. The gradient boosting decision tree prediction method for the sandstone drillability based on the crystal structure and the mineralogical characteristics according to claim 1, wherein a process of training the gradient boosting decision tree model through the training set to obtain the trained gradient boosting decision tree model comprises:
using a decision tree as an initial model for the training set, calculating a residual between a true value of each sample and an initial predicted value, using the residual as a target variable to construct a new decision tree, giving a learning rate, multiplying the learning rate by a predicted value of the new decision tree as a target value increment, adding the initial predicted value and the target value increment to obtain a new predicted value, and using the new decision tree to predict until a predetermined iterations is reached, thus obtaining the trained gradient boosting decision tree model.

7. The gradient boosting decision tree prediction method for the sandstone drillability based on the crystal structure and the mineralogical characteristics according to claim 6, wherein a method for detecting the accuracy of the trained gradient boosting decision tree model through the testing set comprises:
inputting the geometric parameters and mineral components of the testing set into the trained gradient boosting decision tree model to obtain a predicted drillability index, and comparing and analyzing the drillability index of the testing set with the new predicted value to obtain the prediction accuracy of the trained gradient boosting decision tree model.
